(19) United States
(12) Patent Application Publication
(10) Pub. No.: US 2026/0103479 A1
(43) Pub. Date: Apr. 16, 2026

(54) PRODRUGS OF OPICAPONE

(71) Applicant: BIAL- PORTELA & CA, S.A., São Mamede Do Coronado (PT)

(72) Inventors: Laszlo Kiss, São Mamede Do Coronado (PT); Jessica Baiget, São Mamede Do Coronado (PT); Alexander Beliaev, São Mamede Do Coronado (PT); Pedro Barrocas, São Mamede Do Coronado (PT); Evgeny Revunov, São Mamede Do Coronado (PT); Tomislav Karoli, São Mamede Do Coronado (PT); Joerg Holenz, São Mamede Do Coronado (PT)

(21) Appl. No.: 18/852,548

(22) PCT Filed: Mar. 31, 2023
(86) PCT No.: PCT/PT2023/050009

§ 371 Date: Sep. 30, 2024

(30) Foreign Application Priority Data
Apr. 1, 2022 (GB) 2204798.9

(51) Int. Cl. C07F 9/6558; A61K 31/675

(52) U.S. Cl. CPC C07F 9/65583; A61K 31/675

(57) ABSTRACT

This invention relates to prodrugs of opicapone, their synthetic intermediates and pharmaceutically acceptable salts thereof. The invention also relates to methods of preparation of prodrugs of opicapone and pharmaceutically acceptable salts thereof. In particular, the invention relates to specific phosphate prodrugs of opicapone, pharmaceutically acceptable salts thereof and their synthetic intermediates, as well as methods of preparing the same. The invention also relates to administration routes for prodrugs of opicapone.

### FIELD OF THE INVENTION

This invention relates to prodrugs of opicapone, their synthetic intermediates and pharmaceutically acceptable salts thereof. The invention also relates to methods of preparation of prodrugs of opicapone and pharmaceutically acceptable salts thereof. In particular, the invention relates to specific phosphate prodrugs of opicapone, pharmaceutically acceptable salts thereof and their synthetic intermediates, as well as methods of preparing the same. The invention also relates to administration routes for prodrugs of opicapone.

### BACKGROUND OF THE INVENTION

Levodopa (L-DOPA) has been used in clinical practice for several decades in the symptomatic treatment of various conditions, including Parkinson's disease. L-DOPA is able to cross the blood-brain barrier, where it is then converted to dopamine by the enzyme amino acid decarboxylase (AADC), thus increasing dopamine levels in the brain. However, conversion of L-DOPA to dopamine may also occur in peripheral tissues, possibly causing adverse effects. Therefore, it has become standard clinical practice to co-administer a peripheral AADC inhibitor, such as carbidopa or benserazide, which prevents conversion to dopamine in peripheral tissues. It is also known that inhibitors of the enzyme catechol-O-methyltransferase (COMT) may provide clinical improvements in patients afflicted with Parkinson's disease undergoing treatment with L-DOPA, since COMT catalyses the degradation of L-DOPA to the inactive metabolite 3-O-methyldopa.

Opicapone is a potent and long-acting COMT inhibitor. It is bioactive, bioavailable and exhibits low toxicity. This in part is due to the pyridine N-oxide, which is unusual in active pharmaceutical ingredients due to its supposed high chemical and biological reactivity. Opicapone has potentially valuable pharmaceutical properties in the treatment of some central and peripheral nervous system disorders where inhibition of COMT may be of therapeutic benefit, such as, for example, mood disorders; movement disorders, such as Parkinson's disease, parkinsonian disorders and restless legs syndrome; gastrointestinal disturbances; oedema formation states; and hypertension. The development of opicapone is described in L. E. Kiss et al, J. Med. Chem., 2010, 53, 3396-3411 and it was approved, in combination with L-DOPA, for the treatment of Parkinson's disease in the EU in June 2016 and the US in April 2020.

WO 2007/013830 is the first disclosure of opicapone and relates to its use in the treatment of central and peripheral nervous system disorders. WO 2008/094053 discloses that opicapone has good pharmacokinetic properties allowing it to be dosed once daily. WO 2013/089573 discloses that micronization can be used to provide good oral bioavailability. Given the size of the improvement, opicapone is manufactured and marketed in a micronized crystalline form. However, whilst opicapone can be used to treat central and peripheral nervous system disorders by oral administration of daily dosages of 25 or 50 mg, efforts continue to maximise its solubility, absorbance and distribution whilst minimising its metabolism and excretion thereby improving its pharmacokinetic profile.

As WO 2007/013830 teaches that the reduced pyridine analogues are more toxic, it is important to prevent opicapone from being reduced to this metabolite.

Water soluble derivatives of other COMT inhibitors containing a nitrocatechol moiety have been considered. Leppanen. J. et al., Bioorg. Med. Chem. Lett. 10 (2000), 1967-1969, described the synthesis of water soluble derivatives of entacapone. In particular, it disclosed the synthesis of entacapone monophosphate via the reaction of entacapone with phosphorous oxychloride in dry pyridine. However, the yield was poor (45%), a number of common synthetic strategies failed and the phosphorylation site was not confirmed. Furthermore, conversion of the prodrug recovered only ˜60% entacapone with the rest converting to the active metabolite Z-entacapone. Perhaps for these reasons, no prodrug form of entacapone has be developed into a viable medicament in the 20 years since this disclosure. WO 2019/195761 describes various prodrugs of tolcapone. However, most are uncharged to ensure retention in the eye and permeability of the cornea. WO 2019/195761 discloses a phosphate derivative of tolcapone when produced using phosphorous oxychloride in THF and pyridine. The compound is not characterised and the yields and purities are not disclosed. WO 2019/195761 is directed to topical administration of the prodrugs in treating presbyopia and cataracts. Accordingly, it is unrelated to the use of COMT inhibitors to treat central and peripheral nervous system disorders, which does not appear amenable to a prodrug approach. Therefore, before the present invention, it was unclear if prodrug approaches would be applicable to COMT inhibitors for use in treating central and peripheral nervous system disorders, or if a specific phosphate derivative of opicapone could be synthesised reliably.

WO 2021/182981 identified specific “sheaf agglomerates” that can negatively impact on bioavailability of individual preparations of micronized crystalline opicapone and disclosed methods to detect and remove sheaf agglomerates. WO 2022/025781 disclosed kinetically soluble and bioavailable solid dispersions of opicapone.

Therefore, maximising the bioavailability of opicapone remains an active area of research and there remains a need for stable forms of opicapone which have improved bioavailability and which can be reliably synthesised in good yields.

### SUMMARY OF THE INVENTION

The present inventors have identified particular derivatives of opicapone with improved solubility compared to opicapone per se. For example, certain specific phosphate derivatives of opicapone and pharmaceutically acceptable salts thereof.

The phosphate derivatives of the present invention, and their pharmaceutically acceptable salts, display improved solubility in water compared to opicapone itself. Preferably, the phosphate derivatives of opicapone, and pharmaceutically acceptable salts thereof, exhibit at least a 100-fold increase in solubility in pure water (mg/ml) compared to opicapone itself. Furthermore, the phosphate derivatives of opicapone, and pharmaceutically acceptable salts thereof, are preferably stable in potassium buffer solutions at pH=7.4, rat/human plasma and rat/human liver S9 fraction (i.e., the supernatant fraction obtained from liver by centrifuging at 9000 g containing both cytosol and microsomes), but readily converted to opicapone in rat liver homogenate. Thus, the phosphate derivatives of opicapone, and their pharmaceutically acceptable salts, may act as prodrugs of opicapone in vivo.

The present inventors have also identified methods of synthesising specific water-soluble prodrugs of opicapone, and pharmaceutically acceptable salts thereof.

The inventors discovered that the methods employed in the prior art for the synthesis of water-soluble prodrugs of COMT inhibitors are unsuitable for the synthesis of a phosphate prodrug of opicapone. This is due to reaction of phosphorus oxychloride (POCl₃) with the pyridine N-oxide moiety of opicapone. It is reported in the literature that 2-picoline-N-oxides can be converted to the corresponding 2-(chloromethyl)pyridine derivatives using POCl₃ (Redl, S. et al., J. Het. Chem., 43(6), 1447-1453; 2006).

The present inventors have also identified that different prodrugs of opicapone have distinct pharmacokinetic properties depending on the route of administration.

Accordingly, in a first general embodiment, the invention provides a compound of formula (I):

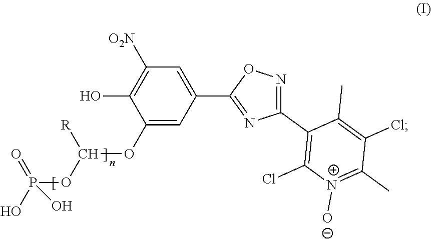

or a pharmaceutically acceptable salt thereof;
wherein R is H or C₁-C₆ alkyl and n is 0 or 1.

In a second general embodiment, the invention provides a method of preparing a compound of formula (I) as set forth above or a pharmaceutically acceptable salt thereof, comprising deprotecting a compound of formula (II):

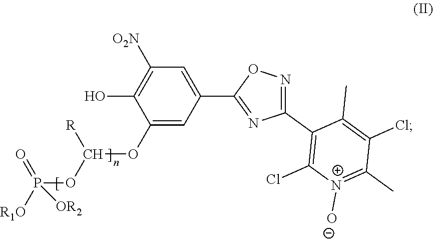

wherein R₁ and R₂ are each independently a monovalent phosphate protecting group or together form a divalent phosphate protecting group; R is H or C₁-C₆ alkyl and n is 0 or 1.

In a third general embodiment, the invention provides a method of preparing a compound of formula (II) as set forth above, which comprises reacting opicapone, i.e., a compound of formula (III):

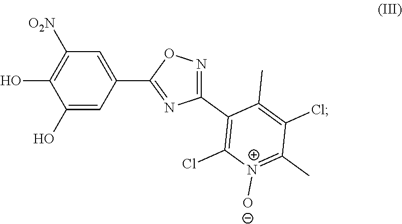 [SMILES: Cc1c(Cl)c(C)[n+]([O-])c(Cl)c1-c1noc(-c2cc(O)c(O)c([N+](=O)[O-])c2)n1]

with a compound of formula (IV):

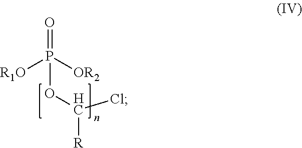

wherein R₁ and R₂ are each independently a monovalent phosphate protecting group or together form a divalent phosphate protecting group, in the presence of a base and an aprotic solvent; R is H or C₁-C₆ alkyl and n is 0 or 1.

In a fourth general embodiment, the invention provides a compound of formula (II) as set forth above, wherein R₁ and R₂ are each independently a monovalent phosphate protecting group or together form a divalent phosphate protecting group; R is H or C₁-C₆ alkyl and n is 0 or 1.

In a fifth general embodiment, the invention provides a pharmaceutical formulation for intravenous administration to a human being comprising:
a. a compound of formula (I) as set forth above or a pharmaceutically acceptable salt thereof; and
b. a pharmaceutically acceptable vehicle.

In a sixth general embodiment, the invention provides a pharmaceutical formulation for subcutaneous administration to a human being comprising:
a. a compound of formula (I) as set forth above or a pharmaceutically acceptable salt thereof; and
b. a pharmaceutically acceptable vehicle.

In a seventh general embodiment, the invention provides a compound of formula (I) or a pharmaceutically acceptable salt thereof, or a pharmaceutical formulation of a compound of formula (I) or a pharmaceutically acceptable salt thereof, for use in the treatment of Parkinson's disease, preferably wherein the compound of formula (I), or a pharmaceutically acceptable salt thereof, is administered in a daily dose which is equivalent to less than 50 mg/day of opicapone, preferably less than 25 mg/day of opicapone.

### BRIEF DESCRIPTION OF THE DRAWINGS

The invention will now be described in detail with reference to the accompanying drawings, in which:

FIG. 1 shows the stability of compound 1 and compound 4 in different matrixes. FIG. 1a shows the clearance of compound 1 in different matrixes (buffer ●; rat liver homogenate ▪; human plasma ▾; rat plasma ♦; human liver S9 fraction ∘) over a 1 hr time period, with measurements being taken at 0 h, 0.1 h, 0.25 h, 0.5 h, 0.75 h and 1 h. Compound concentration=1 μM. FIG. 1b shows the clearance of compound 1 (●) and compound 4 (▪) in rat liver homogenate over a 1 hr time period, with measurements being taken at 0 h, 0.1 h, 0.25 h, 0.5 h, 0.75 h and 1 h. Compound concentration=1 μM.

FIG. 2 shows the stability of compound 1 and compound 4 at different pH values (2500 ng/mL at pH values of 1.2 (●), 4.5 (▪) and 6.8 (▾) over 4 hours at 37° C.). FIG. 2a shows compound 1 was stable in buffer at pH 1.2, pH 4.5 and 6.8, but required some time to solubilise at pH 1.2. FIG. 2b shows compound 4 was stable in buffer at pH 1.2 (●), 4.5 (▪) and 6.8 (▾).

FIG. 3 shows the oral pharmacokinetic properties of compound 1 and compound 4 (3 mg/kg; 0.2% HPMC) in rats compared to micronized crystalline opicapone. FIG. 3a shows compound 1 (●) demonstrated improved pharmacokinetic properties compared to compound 4 (▪). FIG. 3b shows both compound 1 (●) and compound 4 (▪) were converted into opicapone with compound 1 producing an opicapone pharmacokinetic profile comparable to opicapone itself. Compound 4 demonstrated worst oral pharmacokinetic parameters compared to compound 1 and opicapone.

FIG. 4 shows the intravenous pharmacokinetic properties of compound 1 and compound 4 (1 mg/kg; DMSO:20% HPBCD (1:9)) in rats compared to micronized crystalline opicapone. FIG. 4a shows compound 1 (●) demonstrated improved pharmacokinetic properties compared to compound 4 (▪). FIG. 4b shows both compound 1 (∘) and compound 4 (□) were converted into opicapone with compound 1 producing an opicapone pharmacokinetic profile comparable to opicapone itself (▴). Compound 4 demonstrated worst intravenous pharmacokinetic parameters compared to compound 1 and opicapone.

FIG. 5 shows the subcutaneous pharmacokinetic properties of compound 1 and compound 4 (3 mg/kg; DMSO:20% HPBCD (1:9)) in rats compared to oral pharmacokinetic properties of micronized crystalline opicapone. FIG. 5a shows compound 1 (●) was converted into opicapone (∘) producing an opicapone pharmacokinetic profile with improved exposure (about 3-fold increase) when compared to opicapone itself (▴). FIG. 5b shows compound 4 (▪) was converted into opicapone (□) producing an opicapone pharmacokinetic profile with improved exposure (about 3-fold increase) when compared to opicapone itself (▴).

FIG. 6 shows the pharmacodynamic properties of compound 1 and compound 4 on COMT inhibition. Conversion of compound 1 (●) and compound 4 (▪) into opicapone produced a rapid inhibition of COMT activity in erythrocytes, with a maximal effect 2 h after dosing (96% and 94% inhibition for compound 1 and compound 4, respectively). Inhibition of COMT maintained above 80% at least until 8 h post-dosing. The COMT inhibition of compound 1 and compound 4 is higher and more sustained than orally administered opicapone (※).

FIG. 7 shows the pharmacodynamic properties of compound 4 on L-DOPA bioavailability and 3-OMD production compared to a vehicle. FIG. 7a shows the pharmacodynamic properties of compound 4 (▪) on L-DOPA bioavailability compared to a vehicle (□). FIG. 7b shows the pharmacodynamic properties of compound 4 (▪) on 3-OMD production compared to a vehicle (▪). Concomitant administration in a single administration of compound 4 with L-DOPA/benserazide increased circulating levels of L-DOPA (1.7-fold increase in the AUCₗₐₛₜ) with a corresponding decrease in the levels of its metabolite.

### DETAILED DESCRIPTION OF THE INVENTION

### A. Definitions

The following definitions apply to the terms as used throughout this specification unless otherwise limited in specific instances.

The term “prodrug” refers to a compound with no pharmacological activity that is converted to a compound with a desirable pharmacological activity in vivo by enzymatic and/or chemical reactions, which compound then exerts a desirable pharmacological effect.

The term “protecting group” refers to a chemical moiety introduced onto a functional group to block its reactivity under the synthetic conditions needed to make modifications elsewhere on the molecule. The protecting group is stable to certain chemical conditions, but can be facilely removed under specific conditions in a process known as “deprotection”.

The term “phosphate protecting group” refers to a protecting group, as described above, that has been introduced onto a phosphate functional group. Common examples of moieties used as phosphate protecting groups are C₁-C₆ alkyl and benzyl groups.

The term “C₁-C₆ alkyl” means a monovalent unsubstituted saturated straight-chain or branched-chain hydrocarbon radical having from 1 to 6 carbon atoms. “C₁-C₂ alkyl”, “C₁-C₃ alkyl”, “C₁-C₄ alkyl” and “C₁-C₅ alkyl” have analogous meanings. Common examples used as phosphate protecting groups include methyl (C₁) and tert-butyl (C₄).

The term “pharmaceutically acceptable salt” means a salt such as those described in standard texts on salt formation, see for example: P. Stahl, et al., Handbook of Pharmaceutical Salts: Properties, Selection and Use (VCHA/Wiley-VCH, 2002), or S. M. Berge, et al., “Pharmaceutical Salts” (1977) Journal of Pharmaceutical Sciences, 66, 1-19.

The term “pharmaceutically acceptable excipient” means any ingredient of a pharmaceutical composition other than the compound(s) of the invention, or other known pharmacologically active components. The choice of excipient will to a large extent depend on factors such as the particular mode of administration, the effect of the excipient on solubility and stability, and the nature of the dosage form.

The term “vehicle” means a carrier or inert medium used as a solvent in which the medicinally active agent is formulated and or administered in the form of a liquid (Dictionary of Pharmacy, 1986). The term “pharmaceutically acceptable vehicle” means any vehicle that is useful in preparing a liquid pharmaceutical formulation that is generally compatible with the other ingredients of the formulation and, when administered to a human, does not produce an adverse reaction. It comprises a solvent (e.g., water) and, optionally, one or more pharmaceutically acceptable excipients (e.g., buffer(s), surfactant(s), preservative(s) etc.).

The terms “therapy”, “treatment” and “treating” include both preventative and curative treatment of a condition, disease or disorder. It also includes slowing, interrupting, controlling or stopping the progression of a condition, disease or disorder. It also includes preventing, curing, slowing, interrupting, controlling or stopping the symptoms of a condition, disease or disorder.

Where one therapeutic agent is required to be administered “in combination with” another therapeutic agent, this means they must be administered in such a way that both therapeutic agents are present in the patient's body at the same time. The two agents may be administered simultaneously or subsequently, as a single preparation or as separate preparations and via the same or different routes of administration.

The “effective daily dose” of a compound is the total amount of that compound which must be administered each day in order to provide the desired pharmacological (and therefore therapeutic) effect throughout the entire period of treatment. The effective daily dose may be administered as one or more discrete doses which together add up to the effective daily dose, or it may be administered as a continuous infusion.

### B. Water Soluble Prodrugs of Opicapone and Precursors Thereof

The present invention relates to a compound of formula (I):

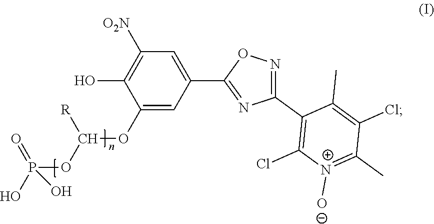

or a pharmaceutically acceptable salt thereof; wherein R is H or C₁-C₆ alkyl and n is 0 or 1. The present inventors discovered that a compound of formula (I) acts as a prodrug of opicapone by remaining stable in buffered solution yet converting back to opicapone in rat liver homogenate.

In one embodiment, the invention relates to a compound of formula (I) or a pharmaceutically acceptable salt thereof; wherein n is 0.

In another embodiment, the invention relates to a compound of formula (I) or a pharmaceutically acceptable salt thereof; wherein n is 1. In a preferred embodiment wherein n is 1, R is H. In an alternative embodiment wherein n is 1, R is C₁-C₆ alkyl, preferably C₁-C₄ alkyl, more preferably C₁-C₂ alkyl, even more preferably methyl.

Pharmaceutically acceptable salts of the compound of formula (I) are preferably selected from the group consisting of a sodium salt, an ammonium salt or a potassium salt. In a more preferred embodiment, the pharmaceutically acceptable salts thereof are selected from the group consisting of a sodium salt and an ammonium salt. For example, the pharmaceutically acceptable salt thereof is a sodium salt. For example, the pharmaceutically acceptable salt thereof is an ammonium salt. In a more preferred embodiment, the sodium salt is a disodium salt and the ammonium salt is a triammonium salt. These salts of opicapone prodrugs were found to be stable, soluble and non-toxic.

The compound of formula (I) may be prepared from a compound of formula (II):

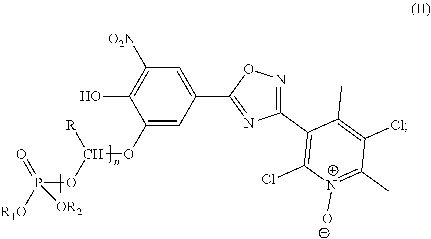

wherein R₁ and R₂ are each independently a monovalent phosphate protecting group or together form a divalent phosphate protecting group; R is H or C₁-C₆ alkyl and n is 0 or 1. The inventors surprisingly discovered that the prior art methods of phosphorylating entacapone and tolcapone were not suitable for directly phosphorylating opicapone to provide a compound of formula (I). However, by first forming the intermediate compound of formula (II), the compound of formula (I) could be isolated in high yield and high purity without modification of the key pyridine N-oxide moiety.

In a preferred embodiment, the phosphate protecting groups are each independently selected from the group consisting of C₁-C₆ alkyl and benzyl. In a more preferred embodiment, the phosphate protecting groups are the same and are selected from the group consisting of methyl, tert-butyl and benzyl. In an alternative more preferred embodiment, the phosphate protecting groups are the same and are ethyl. In an even more preferred embodiment, the phosphate protecting groups are both benzyl or both tert-butyl.

### C. Methods of Synthesising Water-Soluble Prodrugs of Opicapone and Pharmaceutically Acceptable Salts Thereof

The present invention also relates to a method of preparing a compound of formula (I) or a pharmaceutically acceptable salt thereof, which comprises (a) deprotecting a compound of formula (II) as defined above so as to provide a compound of formula (I), and (b) optionally converting the compound of formula (I) into a pharmaceutically acceptable salt.

The deprotection step can be carried out using any one of the following methods: deprotection using a boron agent, such as boron tribromide; deprotection using a trimethylsilyl halide; deprotection via hydrolysis using a strong acid, such as hydrogen bromide (HBr) or hydrochloric acid (HCl); deprotection via oxidation using an oxidant such as cerium ammonium nitrate (CAN); or deprotection via catalytic hydrogenolysis, wherein the catalyst is selected from the group consisting of palladium on charcoal or platinum(IV) oxide. For example, deprotection using a boron agent. For example, deprotection using a trimethylsilyl halide. For example, deprotection via hydrolysis using a strong acid, such as hydrogen bromide (HBr) or hydrochloric acid (HCl). For example, deprotection via catalytic hydrogenolysis, wherein the catalyst is selected from the group consisting of palladium on charcoal or platinum(IV) oxide. In a preferred embodiment, the deprotection step is carried out using trimethylsilyl halide. In a more preferred embodiment, the trimethylsilyl halide is trimethylsilyl bromide.

In a preferred embodiment, the method of preparing a compound of formula (I) comprises deprotection of the compound of formula (II) using trimethylsilyl halide, in an aprotic solvent, followed by aqueous work up. In a more preferred embodiment, the trimethylsilyl halide is trimethylsilyl bromide. In a preferred embodiment, the aprotic solvent may be selected from the group consisting of dichloromethane and acetonitrile. In a more preferred embodiment, the aprotic solvent is dichloromethane. In a most preferred embodiment, the trimethylsilyl halide is trimethylsilyl bromide and the aprotic solvent is dichloromethane.

Optimum reaction temperatures depend on the structure of R₁, R₂, R and n in the compound of formula (II). Preferably, the reaction temperature is in the range of 10° C. to 25° C. In a more preferred embodiment, the reaction temperature is at a controlled temperature of 20 to 25° C., more preferably at a room temperature (rt) of 25° C.

Preferably, the compound of formula (I) is purified by reversed phase chromatography, for example, using a C18 reserved phase column and a water/acetonitrile gradient for the mobile phase. Alternatively, the compound of formula (I) (e.g., compound 1) is converted to the equivalent amine salt (e.g., compound 3) by the addition of aqueous ammonia (e.g. 30% aqueous ammonia) to precipitate the product. The pure amine salt (e.g., compound 3) may be converted to the compound of formula (I) (e.g., compound 1) by the addition of strong acid (e.g., HCl).

The present invention also extends to a method of preparing pharmaceutically acceptable salts of the compound of formula (I).

In one embodiment, the present invention relates to a method of preparing a sodium salt of the compound of formula (I) comprising reacting the compound of formula (I) with sodium hydroxide in a suitable solvent system. For example, a suitable solvent system is ethanol.

In one embodiment, the present invention relates to a method of preparing an ammonium salt of the compound of formula (I) comprising reacting the compound of formula (I) with ammonia in a suitable solvent system. For example, a suitable solvent system is methanol and diethyl ether. Alternatively, a suitable solvent system is isopropanol and water and the ammonium salt of the compound of formula (I) is collected as a precipitate.

### D. Methods of Synthesising Useful Intermediates in the Synthesis of Water-Soluble Prodrugs of Opicapone and Pharmaceutically Acceptable Salts Thereof

The present invention relates to a method of preparing a compound of formula (II), comprising reacting a compound of formula (III):

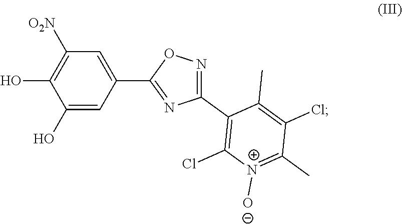 [SMILES: Cc1c(Cl)c(C)[n+]([O-])c(Cl)c1-c1noc(-c2cc(O)c(O)c([N+](=O)[O-])c2)n1]

with a compound of formula (IV):

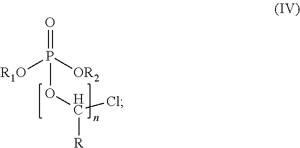

wherein R₁ and R₂ are each independently a monovalent phosphate protecting group or together form a divalent phosphate protecting group; R is H or C₁-C₆ alkyl and n is 0 or 1; in the presence of a base and an aprotic solvent.

In a preferred embodiment, the phosphate protecting groups are each independently selected from the group consisting of C₁-C₆ alkyl and benzyl. In a more preferred embodiment, the phosphate protecting groups are the same and selected from the group consisting of methyl, tert-butyl and benzyl. In an alternative more preferred embodiment, the phosphate protecting groups are the same and are ethyl. In an even more preferred embodiment, the phosphate protecting groups are both benzyl or both tert-butyl.

Preferably, the compound of formula (II) is purified by column chromatography, for example, using a silica stationary phase and a methanol/dichloromethane mixture as the mobile phase.

In a further embodiment the base may be selected from the group consisting of trimethylamine or Hunig's base (N,N-diisopropylethylamine). In a preferred embodiment, the base is trimethylamine (TEA).

In a further embodiment, the aprotic solvent may be selected from the group consisting of dichloromethane, tetrahydrofuran, acetonitrile and ethyl acetate. In a preferred embodiment, the aprotic solvent is dichloromethane.

Preferably, the reaction temperature is in the range of 10° C. to 25° C. In a more preferred embodiment, the reaction temperature is at a controlled temperature of 20 to 25° C., more preferably at a room temperature (rt) of 25° C.

As will be evident to the skilled person, the method of preparing a compound of formula (II), comprising reacting a compound of formula (III) with a compound of formula (IV), can be directly combined with method of preparing a compound of formula (I) or a pharmaceutically acceptable salt thereof, comprising deprotecting a compound of formula (II), and optionally converting the product into a pharmaceutically acceptable salt. In the combined method, the compound of formula (II) may be purified by column chromatography prior to deprotection.

In the methods described above, R₁ and R₂ are preferably each independently selected from the group consisting of C₁-C₆ alkyl and benzyl. In a more preferred embodiment, the phosphate protecting groups are the same and selected from the group consisting of methyl, tert-butyl and benzyl. In an alternative more preferred embodiment, the phosphate protecting groups are the same and are ethyl. In an even more preferred embodiment, the phosphate protecting groups are both benzyl or both tert-butyl.

In one embodiment, the invention relates to a method of preparing a compound of formula (II), comprising reacting a compound of formula (III) with a compound of formula (IV); wherein n is 0.

In another embodiment, the invention relates to a method of preparing a compound of formula (II), comprising reacting a compound of formula (III) with a compound of formula (IV); wherein n is 1. In a preferred embodiment wherein n is 1, R is H. In an alternative embodiment wherein n is 1, R is C₁-C₆ alkyl, preferably C₁-C₄ alkyl, more preferably C₁-C₂ alkyl, even more preferably methyl.

### E. Pharmaceutical Formulations of Water-Soluble Prodrugs of Opicapone and Pharmaceutically Acceptable Salts Thereof

The present invention relates to a pharmaceutical formulation of a compound of formula (I) or a pharmaceutically acceptable salt thereof and one or more pharmaceutically acceptable excipients. The pharmaceutical formulation may be in the form of a solid, for example a tablet or capsule, or in the form of a liquid or in the form of a semi-solid, for example a gel.

In a preferred embodiment, the present invention relates to a pharmaceutical formulation for intravenous, subcutaneous, intraperitoneal or intraduodenal administration (preferably to a human being), more preferably intravenous or subcutaneous administration, most preferably subcutaneous administration comprising;
a. a compound of formula (I), or a pharmaceutically acceptable salt thereof, as set forth above; and
b. a pharmaceutically acceptable vehicle.

In a preferred embodiment, the pharmaceutically acceptable vehicle is an aqueous solution. For example, the pharmaceutically acceptable vehicle may comprise water optionally together with one or more water-miscible solvents such as ethanol, propylene glycol, polyethylene glycol, transcutol, glycerol or DMSO. The aqueous solution may also comprise one or more additives selected from the group consisting of NaCl (saline) and buffers (e.g., phosphate buffer or sodium bicarbonate), to form isotonic solutions with a neutral pH (pH 5 to 7) suitable for intravenous or subcutaneous administration (e.g., to a human), preferably suitable for subcutaneous administration (e.g., to a human).

In a specific embodiment, the present invention relates to a pharmaceutical formulation for intravenous or subcutaneous (preferably subcutaneous) administration comprising a compound of formula (I); preferably compound 1 or compound 4 (more preferably compound 4) and a pharmaceutically acceptable vehicle. The formulation may further comprise L-DOPA and/or carbidopa (preferably L-DOPA and carbidopa). Alternatively, the formulation may further comprise foslevodopa and/or foscarbidopa (preferably foslevodopa and foscarbidopa).

### F. Therapeutic Use

The present invention also relates to compounds of formula (I), pharmaceutically acceptable salts thereof and pharmaceutical formulations of compounds of formula (I) and pharmaceutically acceptable salts thereof, as set forth above, for use in the treatment of Parkinson's disease.

In a preferred embodiment, the compounds or pharmaceutical formulations are administered in combination with L-DOPA or foslevodopa, more preferably in combination with L-DOPA or foslevodopa and a peripheral AADC inhibitor, such as carbidopa, foscarbidopa or benserazide. The L-DOPA or foslevodopa and/or AADC inhibitor may be administered separately or in combination with each other. Where the compound of formula (I), or pharmaceutically acceptable salt thereof, is administered in the form of a solid, for example a tablet or capsule, it may be administered together with the L-DOPA or foslevodopa and/or AADC inhibitor, but it is preferably administered separately from the L-DOPA or foslevodopa and/or AADC inhibitor. Where the compound of formula (I) is administered in the form of an intravenous, subcutaneous or intraperitoneal liquid (preferably an intravenous or subcutaneous liquid, more preferably a subcutaneous liquid), it may be administered separately from or concomitantly (in a single daily administration or multiple daily administrations or continuous administration) with the L-DOPA or foslevodopa and/or AADC inhibitor. Where the compound of formula (I) is administered in the form of an intraduodenal liquid, suspension or semi-solid (example a gel, preferably an intraduodenal gel or suspension, more preferably an intraduodenal gel), it may be administered separately from or concomitantly with the L-DOPA or foslevodopa and/or AADC inhibitor.

In one embodiment, the compound of formula (I) or a pharmaceutically acceptable salt thereof, is administered in an effective daily dose which is equivalent to less than 50 mg/day of opicapone. However, the compound of formula (I) or a pharmaceutically acceptable salt thereof, may be administered in an effective daily dose which is equivalent to 50 mg/day of opicapone. In a preferred embodiment, the compound of formula (I) has an effective daily dose which is equivalent to 1 to 30 mg/day of a compound of opicapone, more preferably equivalent to 2 to 25 mg/day, even more preferably equivalent to 5 to 20 mg/day. In this context, the word “equivalent” means that the daily dosage contains less than 50 mg of opicapone when the phosphate moiety and any associated counter-ions of the pharmaceutically acceptable salt thereof are excluded on a mass basis.

The effective daily dosage may be administered in discrete dosages (solid, liquid or semi-solid, preferably a liquid) or as a continuous liquid or semi-solid infusion.

Subcutaneous injection has the advantages that it is suitable for continuous administration, but is less invasive compared to intravenous or intraduodenal administration. This can result in improved patient wellbeing and/or compliance with the treatment. However, the bioavailability of specific compounds or prodrugs via a particular route cannot be predicted.

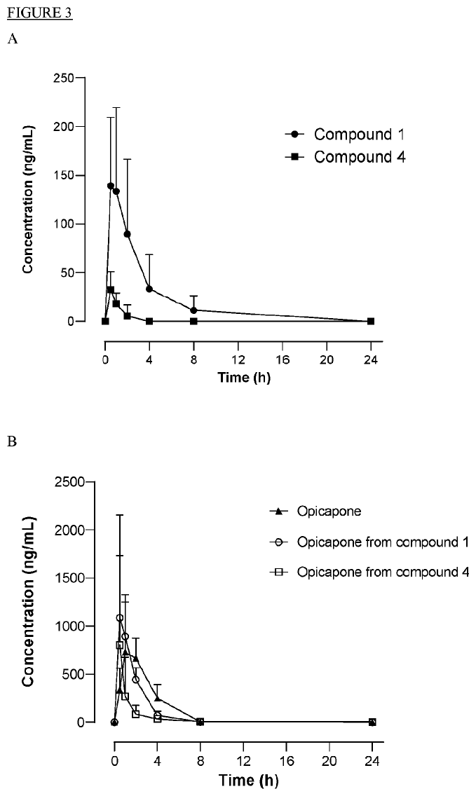

Based on the data shown in FIG. 3 to FIG. 7, intravenous administration is preferred for compounds of formula (I). In particular, intravenous administration is most preferred for compounds of formula (I) wherein n is 0 (e.g., compound 1 or the salts thereof (such as compound 2 or compound 3)). Where the compound of formula (I) is administered in the form of an intravenous liquid, it may be administered separately from or concomitantly with the L-DOPA or foslevodopa and/or AADC inhibitor.

Based on the data shown in FIG. 3 to FIG. 7, subcutaneous administration is preferred for compounds of formula (I). In particular, subcutaneous administration is most preferred for compounds of formula (I) wherein n is 1 (e.g., compound 4). Where the compound of formula (I) is administered in the form of a subcutaneous liquid, it may be administered separately from or concomitantly with the L-DOPA or foslevodopa and/or AADC inhibitor. In a particularly preferred embodiment, the present invention provides compound 4 for use in the treatment of Parkinson's disease, wherein the compound 4 is administered subcutaneously in combination with L-DOPA or foslevodopa, more preferably in combination with L-DOPA or foslevodopa and a peripheral AADC inhibitor, such as carbidopa, foscarbidopa or benserazide.

The L-DOPA and/or AADC inhibitor are preferably administered in the form of an intravenous, subcutaneous or intraperitoneal liquid (preferably a subcutaneous or intravenous liquid, more preferably a subcutaneous liquid). They may themselves be administered in the form of prodrugs. For example, L-DOPA may be administered in the form of foslevodopa (levodopa-4′-monophosphate). For example, the AADC inhibitor may be administered in the form of foscarbidopa (carbidopa 4′-monophosphate). For example, L-DOPA may be administered in the form of foslevodopa and the AADC inhibitor may be administered in the form of foscarbidopa. The foslevodopa and foscarbidopa may be administered in the form a binary composition (e.g., foslevodopa/foscarbidopa; ABBV-951) with the compound of formula (I) administered separately. The foslevodopa, foscarbidopa and the compound of formula (I) may be administered in the form a ternary composition (e.g., foslevodopa/foscarbidopa/compound of formula (I)).

### G. EXAMPLES

### Preparation of Intermediates

### Compound of Formula (IV)—Dibenzyl Phosphorochloridate

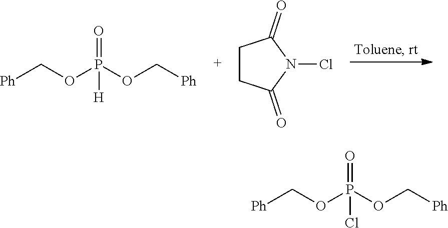 [SMILES: O=[PH](OCc1ccccc1)OCc1ccccc1 ; O=C1CCC(=O)N1Cl ; O=P(Cl)(OCc1ccccc1)OCc1ccccc1]

To a stirred solution of dibenzyl phosphate (1 mL, 1, 187 g, 4.53 mmol) in toluene (20 mL) was added N-chlorosuccinimide. The reaction was stirred at room temperature for 3 h. Thereupon, succinimide was filtered off (through a short pad of celite), and then the filtrate was evaporated to give dibenzyl phosphorochloridate (1.6 g) as a colourless oil which was used in the next step without further purification.

### Compound of formula (III)—2,5-dichloro-3-(5-(3,4-dihydroxy-5-nitrophenyl)-1,2,4-oxadiazol-3-yl)-4,6-dimethylpyridine 1-oxide

The synthesis of opicapone, i.e., the compound of formula (III), is described in WO 2007/013830 or WO 2013/089573. The method described in WO 2013/089573 is preferred.

### Compound of formula (II)—3-(5-(3-((bis(ethoxy)phosphoryl)oxy)-4-hydroxy-5-nitrophenyl)-1,2,4-oxadiazol-3-yl)-2,5-dichloro-4,6-dimethylpyridine 1-oxide

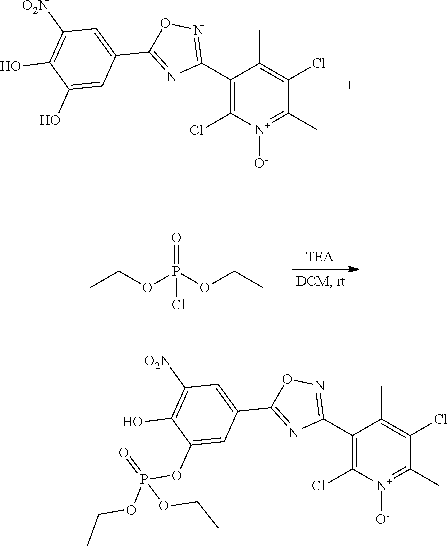

A mixture of 2,5-dichloro-3-(5-(3,4-dihydroxy-5-nitrophenyl)-1,2,4-oxadiazol-3-yl)-4,6-dimethylpyridine 1-oxide (10 g, 24.2 mmol), diethyl phosphorochloridate (4.59 g, 26.6 mmol), and triethylamine (10.12 mL, 72.6 mmol) in dichloromethane (100 mL) was mixed at 0° C., warmed to room temperature and stirred at this temperature overnight. The obtained brown solution was quenched with water (100 mL). The organic layer was washed with HCl 4M solution (2×100 mL), brine, dried over sodium sulphate, filtered, and then evaporated to afford 11.6 g of the product 3-(5-(3-((bis(ethoxy)phosphoryl)oxy)-4-hydroxy-5-nitrophenyl)-1,2,4-oxadiazol-3-yl)-2,5-dichloro-4,6-dimethylpyridine 1-oxide as a yellow solid.

### Compound of formula (II)—3-(5-(3-((bis(benzyloxy)phosphoryl)oxy)-4-hydroxy-5-nitrophenyl)-1,2,4-oxadiazol-3-yl)-2,5-dichloro-4,6-dimethylpyridine 1-oxide

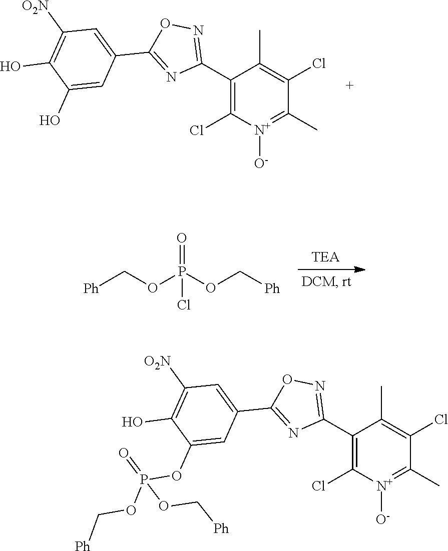 [SMILES: Cc1c(Cl)c(C)[n+]([O-])c(Cl)c1-c1noc(-c2cc(O)c(O)c([N+](=O)[O-])c2)n1 ; O=P(Cl)(OCc1ccccc1)OCc1ccccc1 ; Cc1c(Cl)c(C)[n+]([O-])c(Cl)c1-c1noc(-c2cc(OP(=O)(OCc3ccccc3)OCc3ccccc3)c(O)c([N+](=O)[O-])c2)n1]

A mixture of 2,5-dichloro-3-(5-(3,4-dihydroxy-5-nitrophenyl)-1,2,4-oxadiazol-3-yl)-4,6-dimethylpyridine 1-oxide (1.559 g, 3.77 mmol), dibenzyl phosphorochloridate (1.343 g, 4.53 mmol), and triethylamine (1.577 mL, 11.32 mmol) in dichloromethane (25 mL) was stirred at room temperature overnight. To the obtained brown solution few drops of methanol and water were added. The organic layer was washed with brine, dried over magnesium sulphate, filtered, and then evaporated to give the crude product as an orange solid. Chromatographic purification on silica (in a methanol/dichloromethane mixture) afforded 1.027 g of 3-(5-(3-((bis(benzyloxy)phosphoryl)oxy)-4-hydroxy-5-nitrophenyl)-1,2,4-oxadiazol-3-yl)-2,5-dichloro-4,6-dimethylpyridine 1-oxide as an orange solid.

### Compound of formula (II)—2,5-dichloro-3-(5-(3-(((di-tert-butoxyphosphoryl)oxy)methoxy)-4-hydroxy-5-nitrophenyl)-1,2,4-oxadiazol-3-yl)-4,6-dimethylpyridine 1-oxide

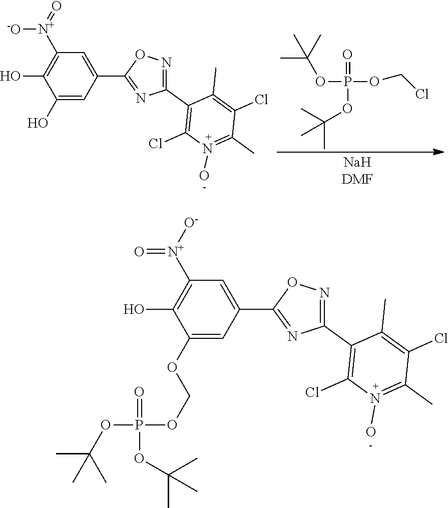

Sodium hydride (17.01 mg, 0.425 mmol) was added to a solution of 2,5-dichloro-3-(5-(3,4-dihydroxy-5-nitrophenyl)-1,2,4-oxadiazol-3-yl)-4,6-dimethylpyridine 1-oxide (160 mg, 0.387 mmol) in DMF (1 mL). After 10 min, tetrabutylammonium iodide (100 mg, 0.271 mmol) and di-tert-butyl (chloromethyl) phosphate (120 mg, 0.464 mmol) in DMF (1 mL) were added and the mixture was stirred at room temperature for 1 h and stirred at 50° C. for 1 h. After cooling down, 5% citric acid in water was slowly added and the solution was extracted with ethyl acetate. The combined organic layer was washed with brine, dried over MgSO₄, filtered and evaporated to dryness. The resulting crude was purified by flash column to obtain a fluffy yellow solid as the title compound. (95 mg, 27%).

### Preparation of Opicapone Prodrugs and Pharmaceutically Acceptable Salts Thereof

### Compound of formula (I)—2,5-dichloro-3-(5-(4-hydroxy-3-nitro-5-(phosphonooxy)phenyl)-1,2,4-oxadiazol-3-yl)-4,6-dimethylpyridine 1-oxide (Compound 1)

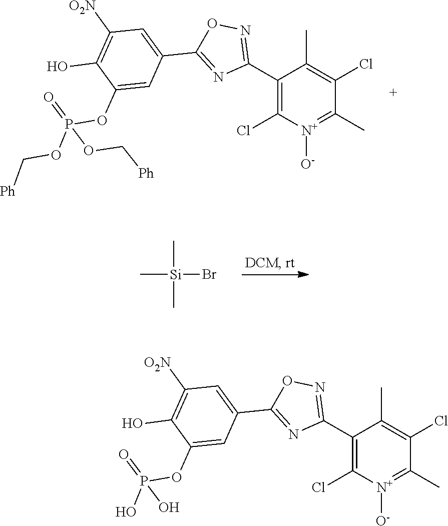

To an ice-cold solution of 3-(5-(3-((bis(benzyloxy)phosphoryl)oxy)-4-hydroxy-5-nitrophenyl)-1,2,4-oxadiazol-3-yl)-2,5-dichloro-4,6-dimethylpyridine 1-oxide (350 mg, 0.520 mmol) was added trimethylsilyl bromide (183 mg, 0.155 mL, 1.195 mmol) under nitrogen atmosphere. After being stirred in the cold for 30 min., the reaction was quenched with few drops of water and then evaporated. Reverse phase chromatographic purification in acetonitrile—water (gradient elution, 0 to 10% acetonitrile) afforded 105 mg of 2,5-dichloro-3-(5-(4-hydroxy-3-nitro-5-(phosphonooxy)phenyl)-1,2,4-oxadiazol-3-yl)-4,6-dimethylpyridine 1-oxide as a pale yellow solid (90% purity, yield: 37%).

¹H NMR (DMSO_{d6}): 8.39 (1H, d, J=2.2 Hz), 8.18 (1H, dd, J=1.2, 2.2 Hz), 2.66 (3H, s), 2.24 (3H, s).

¹³C NMR (DMSO_{d6}): 174.8, 164.6, 150.4, 149.5 (d, J=5.5 Hz), 143.3 (d, J=5.5 Hz), 139.4, 139.3, 134.2, 131.1, 123.3 (d, J=3 Hz), 122.6, 120.3, 112.1, 17.9, 16.5.

### Disodium salt of the compound of formula (I)—5-(3-(2,5-dichloro-4,6-dimethyl-1-oxidopyridin-3-yl)-1,2,4-oxadiazol-5-yl)-2-hydroxy-3-nitrophenyl phosphate, disodium salt (Compound 2)

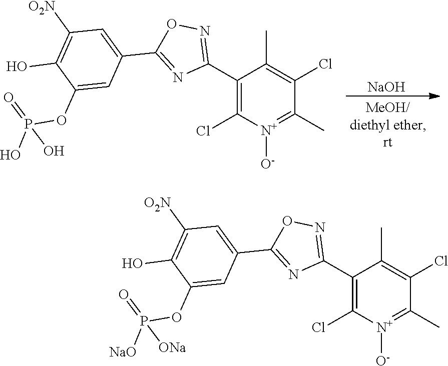

To a solution of 2,5-dichloro-3-(5-(4-hydroxy-3-nitro-5-(phosphonooxy)phenyl)-1,2,4-oxadiazol-3-yl)-4,6-dimethylpyridine 1-oxide (48 mg, 0.097 mmol) in absolute ethanol (3 mL) was added diethyl ether (3 mL) followed by addition of 1M sodium hydroxide (0.195 mL, 0.195 mmol) dropwise with stirring at 20-25° C. After being stirred for 15 min., the obtained solid was collected by filtration, washed with a mixture of ethanol—diethyl ether (1:1), and then dried in vacuum at 40° C. to give sodium 5-(3-(2,5-dichloro-4,6-dimethyl-1-oxidopyridin-3-yl)-1,2,4-oxadiazol-5-yl)-2-hydroxy-3-nitrophenyl phosphate as an orange powder (47 mg, yield: 90%).

¹H NMR (D₂O): 8.37 (1H, d, J=2.4 Hz), 7.83 (1H, dd, J=1.4, 2.4 Hz), 2.60 (3H, s), 2.15 (3H, s)

¹³C NMR (D₂O): 178.4, 165.4, 161.7 (d, J=4 Hz), 153.7, 149.0 (d, J=6 Hz), 142.9, 142.3, 139.5, 134.2, 124.6, 124.4, 123.5 (d, J=3 Hz), 108.0, 19.5, 17.9

### Triammonia salt of the compound of formula (I)—2,5-dichloro-3-(5-(4-hydroxy-3-nitro-5-(phosphonooxy)phenyl)-1,2,4-oxadiazol-3-yl)-4,6-dimethylpyridine 1-oxide, triammonia salt (Compound 3)

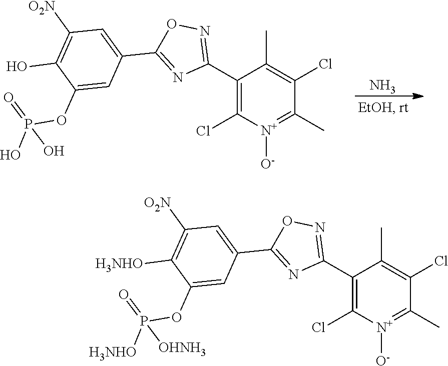

To a stirred solution of 2,5-dichloro-3-(5-(4-hydroxy-3-nitro-5-(phosphonooxy)phenyl)-1,2,4-oxadiazol-3-yl)-4,6-dimethylpyridine 1-oxide (20 mg, 0.041 mmol) in absolute ethanol (1 mL) was added a 2M solution of ammonia (0.101 mL, 0.203 mmol) in methanol. After being stirred for 15 min., the obtained solid was collected by filtration, washed with ethanol, and then dried in vacuum at 40° C. to give 2,5-dichloro-3-(5-(4-hydroxy-3-nitro-5-(phosphonooxy)phenyl)-1,2,4-oxadiazol-3-yl)-4,6-dimethylpyridine 1-oxide, triammonia salt as an orange powder (15 mg, yield: 68%).

¹H NMR (DMSO_{d6}): 8.25 (1H, d, J=2.4 Hz), 7.67 (1H, dd, J=0.7, 2.4 Hz), 6.90 (12H, br), 2.66 (3H, s), 2.23 (3H, s).

¹³C NMR (DMSO_{d6}): 176.7, 164.3 (d, J=5 Hz), 164.2, 150, 149.9 (d, J=7 Hz), 139.4, 137.1, 134.0, 130.9, 123.5, 121.9, 117.8 (d, J=2.5 Hz), 100.2, 17.9, 16.5.

### Triammonia salt of the compound of formula (I) 2,5-dichloro-3-(5-(4-hydroxy-3-nitro-5-(phosphonooxy)phenyl)-1,2,4-oxadiazol-3-yl)-4,6-dimethylpyridine 1-oxide (Compound 3)

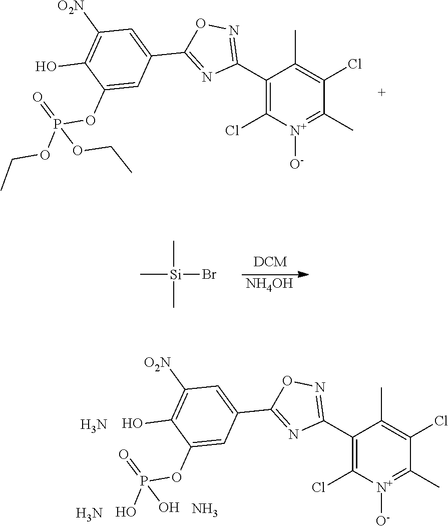

To the solution of 3-(5-(3-((bis(ethoxy)phosphoryl)oxy)-4-hydroxy-5-nitrophenyl)-1,2,4-oxadiazol-3-yl)-2,5-dichloro-4,6-dimethylpyridine 1-oxide (5.00 g, 9.10 mmol) in DCM (100 mL) at −10° C. and nitrogen atmosphere, trimethylsilyl bromide (13.94 g, 12.01 mL, 91 mmol) was added. The mixture was warmed to 40° C. and left stirring at room temperature overnight. The solvent was evaporated, and solids were dissolved in the mixture of MeOH (50 mL) and water (5 mL) and evaporated again. The residues were dissolved in IPA 100 mL, and water (10 mL) and ammonia solution (30% aq., 5 mL) were added to the filtered clear solution, to precipitate the product. The resulting suspension was heated to 70° C. and cooled to 0-5° C. and warmed to room temperature. The solid product was filtered and washed with IPA (2×20 mL), dried in a vacuum at 2 mbar 40° C., afforded 4.2 g of 2,5-dichloro-3-(5-(4-hydroxy-3-nitro-5-(phosphonooxy)phenyl)-1,2,4-oxadiazol-3-yl)-4,6-dimethylpyridine 1-oxide triammonia salt, as an orange solid (90% purity, yield: 85%).

### Compound of formula (I)—Compound 4: 2,5-dichloro-3-(5-(4-hydroxy-3-nitro-5-((phosphonooxy)methoxy)phenyl)-1,2,4-oxadiazol-3-yl)-4,6-dimethylpyridine 1-oxide

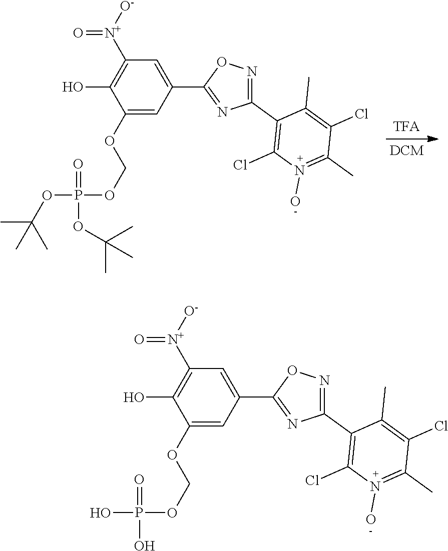

Trifluoroacetic acid (0.108 ml, 1.416 mmol) was added to a solution of 2,5-dichloro-3-(5-(3-(((di-tert-butoxyphosphoryl)oxy)methoxy)-4-hydroxy-5-nitrophenyl)-1,2,4-oxadiazol-3-yl)-4,6-dimethylpyridine 1-oxide (90 mg, 0.142 mmol) in dichloromethane (1.5 ml) at 0° C. The reaction mixture was stirred at room temperature for 45 min. The solvent was evaporated, and the residue was purified by flash chromatography using reverse phase column to obtain a pale-yellow solid as the desired compound (28 mg, 34%).

¹H NMR (DMSO_{d6}): 8.37 (1H, d, J=2.1 Hz), 8.15 (1H, d, J=2.1 Hz), 5.68 (2H, d, J=14.0 Hz), 2.66 (3H, s), 2.25 (3H, s). ¹³C NMR (DMSO_{d6}): 175.0, 164.6, 150.4, 149.3, 147.8, 139.4, 138.7, 134.1, 131.1, 122.7, 120.2, 119.9, 111.9, 90.2 (d, J=4.5 Hz), 17.9, 16.5.

Free form of the compound of formula (I) (e.g., compound 1 or compound 4) can be easily obtained from the related salt via acidification of the related salt.

### Solubility and Stability Studies of Prodrugs of Opicapone

### Solubility Studies

Aqueous solubility studies were completed for compounds 1, 2 and 3, as set forth above, as well as for opicapone per se. The solubility studies were completed at room temperature. For the determination of the solubility increasing amounts of distilled water was added to 5 mg of the test sample until its complete dissolution of the product observed by naked eye. A total of 0.8 mL of distilled water was sufficient to fully solubilize it which corresponds to an approximate solubility of 6 mg/mL.

The results are shown in Table 1 below.

TABLE 1
Solubility of opicapone and opicapone
prodrugs at room temperature

| Compound | Solubility in pure water (mg/mL) |
|---|---|
| Opicapone | 0.023-0.036 |
| Compound 1 | 6 |
| Compound 2 | >0.5 |
| Compound 3 | >0.5 |
| Compound 4 | >0.5 |

The solubility data in Table 1 confirms that compounds 1 to 4 significantly increased solubility compared to opicapone per se in pure water. Compound 1 exhibited a 160-fold increase in solubility in pure water compared to opicapone per se.

### In Vitro Stability Studies

The stability of 1 μM of compound 1 was assessed in the following fractions: phosphate buffer, human plasma, rat plasma, human liver S9 fraction, rat liver S9 fraction and rat liver homogenates.

Reaction was initiated by adding of 1 μM of test compound to the desired matrix (plasma, liver homogenate at 0.4 g/mL or S9 fraction at 1 mg/mL, respectively), with a final volume of 350 μL. At the following time points 0, 5, 15, 30, 45, 60 min, 50 μL of sample is taken and precipitated with 200 μL of acetonitrile 1% formic acid containing the internal standard (opicapone ¹³C₆) at 1 μM. Samples were centrifuged at 20,000 g at 4° C. for 10 min, and then analysed by LC-MS/MS using a bioanalytical method previously developed in-house.

Solutions of the test compounds were prepared at 10 μM using 50 mM potassium phosphate buffer (KPB) at pH 7.4, by adding 1 μL of compound stock at 10 mM to 999 μL KPB 50 mM.

A solution of 100 mM K₂HPO₄ was prepared by weighing 17.42 g and adding to 1 L of MilliQ water. A solution of 100 mM KH₂PO₄ was prepared by weighing 13.61 g and adding to 1 L of MilliQ water. To achieve pH 7.4, ˜250 ml of 100 mM KH₂PO₄ solution was added to 100 mM K₂HPO₄, with continuous measuring of pH. To prepare the solution of potassium phosphate buffer at 50 mM, 500 mL of 100 mM potassium phosphate buffer (pH 7.4) was added to 500 mL MilliQ water.

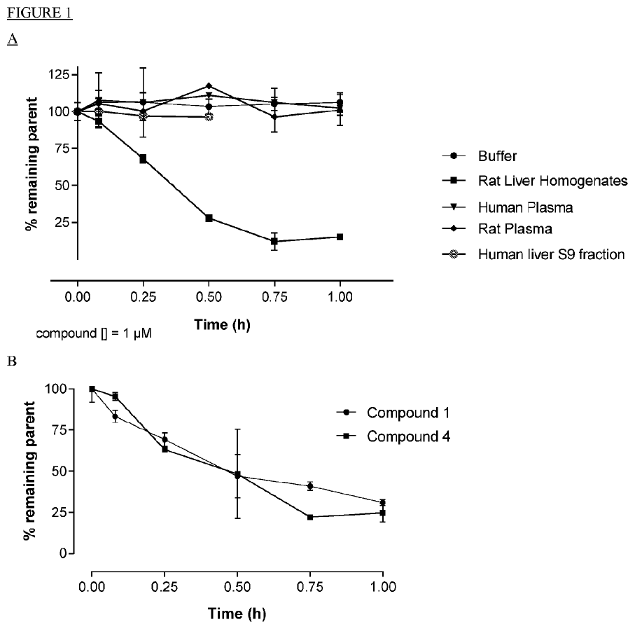

As shown in FIG. 1a, compound 1 was stable in buffer, both human and rat plasma and in both human and rat liver S9 fraction after 0.75 h. However, compound 1 was successfully converted to opicapone in the rat liver homogenates. After 0.5 h the % remaining of the parent compound was less than 25%.

The stability of 1 μM of compound 4 was assessed in the following fractions: phosphate buffer, human plasma, rat plasma, human liver S9 fraction, rat liver S9 fraction and rat liver homogenates.

Reaction was initiated by adding 1 μM of test compound to the desired matrix (plasma or S9 fraction at 1 mg/mL), with a final volume of 250 μL. For plasma, at the time points of 0, 15, 30, 45 and 60 min of incubation, 50 μL of sample is taken and precipitated with 100 μL of internal standard working solution (ISWS) consisting of acetonitrile 1% formic acid containing the internal standard (opicapone ¹³C₆) at 1 μM. For S9 fraction, 50 μL of sample was taken and precipitated with 100 μL of ISWS after 60 min of incubation. For rat liver homogenates (0.2 g/mL of protein), reaction was initiated by adding of 1 μM of test compound with a final volume of 350 μL. At the following time points of 0, 5, 15, 30, 45, 60 min of incubation, 50 μL of sample is taken and precipitated with 200 μL ISWS. Samples were centrifuged at 20,000 g at 4° C. for 10 min, and then analysed by LC-MS/MS using bioanalytical methods previously developed in-house. Compound 4 was stable in all matrices with exception of rat liver homogenates, where the remaining compound was of approximately 31%, after 60 min of incubation. Solutions of the test compounds were prepared at 10 μM using 50 mM potassium phosphate buffer (KPB) at pH 7.4, by adding 1 μL of compound stock at 10 mM to 999 μL KPB 50 mM.

A solution of 100 mM K₂HPO₄ was prepared by weighing 17.42 g and adding to 1 L of MilliQ water. A solution of 100 mM KH₂PO₄ was prepared by weighing 13.61 g and adding to 1 L of MilliQ water. To achieve pH 7.4, ˜250 ml of 100 mM KH₂PO₄ solution was added to 100 mM K₂HPO₄, with continuous measuring of pH. To prepare the solution of potassium phosphate buffer at 50 mM, 500 mL of 100 mM potassium phosphate buffer (pH 7.4) was added to 500 mL MilliQ water.

Compound 4 was stable in buffer, both human and rat plasma and in both human and rat liver S9 fraction after 0.75 h. As shown in FIG. 1b, compound 4 was successfully converted to opicapone in the rat liver homogenates (alongside a repeat experiment for compound 1).

The stability of 2500 ng/mL of compounds 1 and 4 was assessed at different pH values of 1.2, 4.5 and 6.8 over 4 hours at 37° C.
Buffer solutions were prepared as follows:
Buffer solution pH 1.2:
To prepare 1L:
3.73 g potassium chloride
7 mL HCl37%
Add purified water
Check/Adjust pH to 1.20±0.05 (using solutions: HCl 1M and/or NaOH 1M)
Buffer solution pH 4.5
To prepare 1L:
2.99 g sodium acetate trihydrate
14 mL acetic acid 2M
Add purified water
Check/Adjust pH to 4.50±0.05 (using solutions: acetic acid 2M and/or NaOH 1M)
Buffer solution pH 6.8
To prepare 1L:
6.81 g potassium dihydrogen phosphate
22 mL NaOH 1M
Add purified water
Check/Adjust pH to 6.80±0.05 (using solutions: HCl 1M and/or NaOH 1M)

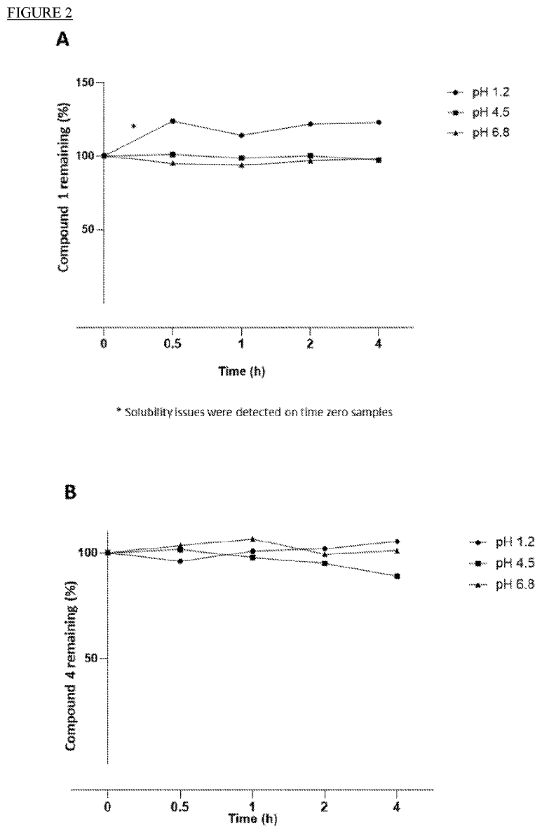

As shown in FIG. 2a, compound 1 was stable in buffer at pH 1.2, pH 4.5 and 6.8, but was not immediately soluble at pH 1.2. As shown in FIG. 2b, compound 4 was stable in buffer at pH 1.2, 4.5 and 6.8.

Long-term stability studies in the solid state indicate compound 4 is particularly stable.

### Pharmacokinetic Properties of Prodrugs of Opicapone

### Oral Pharmacokinetics

The pharmacokinetic properties of compounds 1 and 4 compared to micronized crystalline opicapone were assessed by oral administrations to 4 male Wistar rats (3 mg/kg; 0.2% HPMC).

During the studies, blood was collected at different time points, from tail vein, spun at 1500×g in a refrigerated centrifuge (4° C.) for 15 min, and the plasma obtained was stored at −80° C. until further analysis. The plasma samples collected were analysed for compound 1, compound 4 and opicapone exposure. The bioanalysis involved the use of LC-MS/MS after plasma precipitation.

As shown in FIG. 3a, compound 1 demonstrated improved pharmacokinetic properties compared to compound 4.

As shown in FIG. 3b, both compound 1 and compound 4 were converted into opicapone with compound 1 producing an opicapone pharmacokinetic profile comparable to opicapone itself. Opicapone from compound 4 demonstrated worse oral pharmacokinetic parameters compared to opicapone from compound 1 and opicapone.

### Intravenous Pharmacokinetics

The pharmacokinetic properties of compounds 1 and 4 compared to micronized crystalline opicapone were assessed by intravenous injection to 3 male Wistar rats (1 mg/kg; DMSO:20% HPBCD (1:9).

During the studies, blood was collected at different time points, from jugular vein, spun at 1500×g in a refrigerated centrifuge (4° C.) for 15 min, and the plasma obtained was stored at −80° C. until further analysis. The plasma samples collected were analysed for compound 1, compound 4 and opicapone exposure. The bioanalysis involved the use of LC-MS/MS after plasma precipitation.

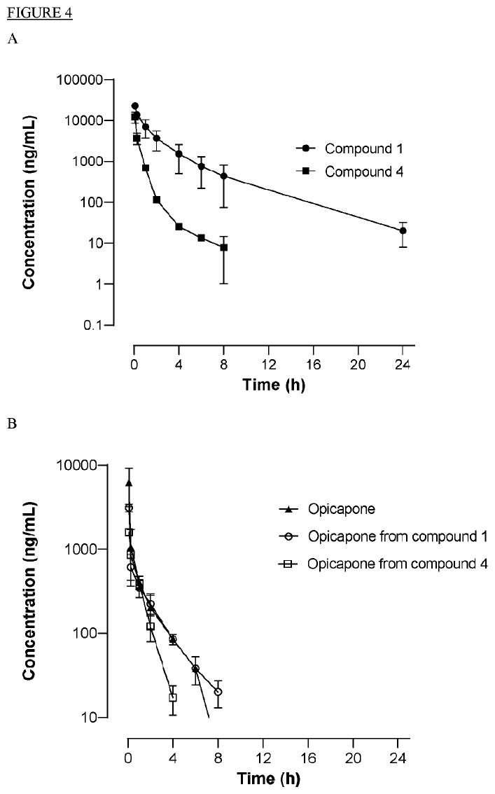

As shown in FIG. 4a, compound 1 demonstrated improved pharmacokinetic properties compared to compound 4.

As shown in FIG. 4b, both compound 1 and compound 4 were converted into opicapone with compound 1 producing an opicapone pharmacokinetic profile comparable to opicapone itself. Opicapone from compound 4 demonstrated worse intravenous pharmacokinetic parameters compared to opicapone from compound 1 and opicapone.

### Subcutaneous Pharmacokinetics

The pharmacokinetic properties of compounds 1 and 4 were assessed by subcutaneous injection to 4 male Wistar rats (3 mg/kg; DMSO:20% HPBCD (1:9)). The compounds showed low levels of irritation at the injection site.

During the studies, blood was collected at different time points, from tail vein, spun at 1500×g in a refrigerated centrifuge (4° C.) for 15 min, and the plasma obtained was stored at −80° C. until further analysis. The plasma samples collected were analysed for compound 1, compound 4 and opicapone exposure. The bioanalysis involved the use of LC-MS/MS after plasma precipitation.

Pharmacokinetic parameters for compound 1 and compound 4 are shown it Table 2.

TABLE 2
Pharmacokinetic parameters for Compound 1 and
Compound 4 administered orally (p.o.), intravenously
(i.v.) and subcutaneously (s.c.)

| | Compound 1 | | | Compound 4 | | |
|---|---|---|---|---|---|---|
| | p.o. | s.c. | i.v. | p.o. | s.c. | i.v. |
| C₀ (ng/mL) | | | 29386.8 | | | 21904.1 |
| Cₘₐₓ (ng/mL) | 145.9 | 7770.4 | | 32.5 | 3141.8 | |
| Tₘₐₓ (h) | 0.5 | 1 | 10.08 | 0.5 | 0.5 | 0.08 |
| AUCₗₐₛₜ | 417.1 | 15452.5 | 30726.7 | 26.5 | 9659.8 | 4985.2 |
| (ng/mL*h) | | | | | | |
| t_{1/2} (h) | | | 2.6 | | | 2.9 |
| CL (mL/h/Kg) | | | 38.6 | | | 214.7 |
| Vd (mL/Kg) | | | 122.6 | | | 1992.5 |

Pharmacokinetic parameters for opicapone converted from compound 1 and compound 4 are shown it Table 3 (alongside those for micronized crystalline opicapone).

TABLE 3
Pharmacokinetic parameters for opicapone converted by the following compounds
administered orally (p.o.), intravenously (i.v.) and subcutaneously (s.c.)

| | Prodrug conversion into Opicapone | | | | | | | |
|---|---|---|---|---|---|---|---|---|
| | Compound 1 | | | Compound 4 | | | Opicapone | |
| | p.o. | s.c. | i.v. | p.o. | S.c. | i.v. | p.o. | i.v. |
| C₀ (ng/mL) | | | 9034.1 | | | 2131.8 | | 15564.7 |
| Cₘₐₓ (ng/mL) | 1117.7 | 1586.2 | | 802.8 | 1854.4 | | 829.6 | |
| Tₘₐₓ (h) | 0.5 | 2 | 0.08 | 0.5 | 2 | 0.08 | 2 | 0.08 |
| AUCₗₐₛₜ | 1995.8 | 6245.6 | 2021.8 | 763.3 | 6951.8 | 1217.2 | 2275.6 | 2865.5 |
| (ng/mL*h) | | | | | | | | |
| t_{1/2} (h) | | | 1.8 | | | 0.7 | | 2.7 |
| CL (mL/h/Kg) | | | 487.1 | | | 816.0 | | 303.9 |
| Vd (mL/Kg) | | | 1292.0 | | | 781.8 | | 1118.6 |

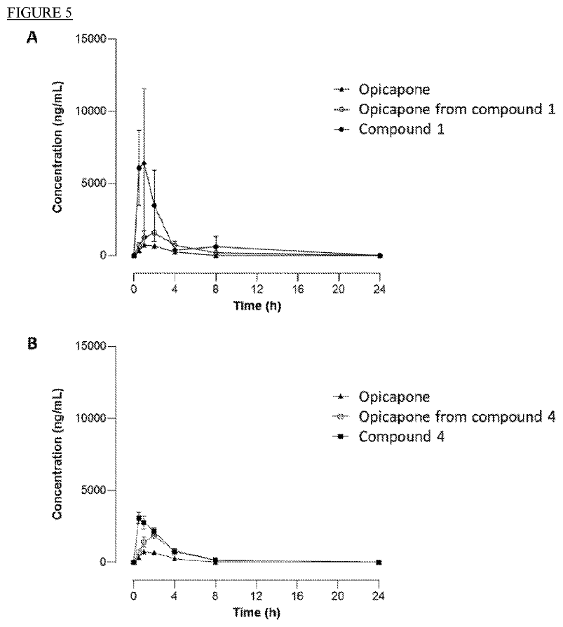

As shown in FIG. 5a, compound 1 was converted into opicapone producing a better opicapone pharmacokinetic profile then opicapone itself after oral administration (about 3-fold increase in AUCₗₐₛₜ). Surprisingly, unlike the oral or intravenous dosing, opicapone from compound 1 displayed an improved pharmacokinetic profile (compared to orally administered opicapone) when administered subcutaneously.

As shown in FIG. 5b, compound 4 was converted into opicapone producing a better opicapone pharmacokinetic profile comparable to opicapone itself after oral administration (about 3-fold increase in AUCₗₐₛₜ). Surprisingly, unlike the oral or intravenous dosing, opicapone from compound 4 displayed an improved pharmacokinetic profile (compared to orally administered opicapone) when administered subcutaneously. This is particularly surprising because opicapone from compound 4 was significantly worse than opicapone or opicapone from compound 1 when administered intravenously or orally. Therefore, opicapone from compound 4 is significantly worse than opicapone from compound 1 when administered intravenously, but with similar pharmacokinetics profile as opicapone from compound 1 when administered subcutaneously. Such unusual pharmacodynamic behaviour could not be predicted by theory. Furthermore, compound 4 produced a higher Cₘₐₓ for opicapone compared to compound 1 despite itself having a lower Cₘₐₓ compared to compound 1.

In combination, the data suggest compound 1 is surprisingly more stable than compound 4 when administered intravenously, suggesting improved stability in the bloodstream. Likewise, compound 1 is more bioavailable via the subcutaneous route due to the higher Cₘₐₓ of the prodrug of compound 1 in FIG. 5a (●) compared to compound 4 in FIG. 5b (▪). Therefore, it is particularly unexpected that more opicapone is produced by compound 4 in FIG. 5b (□) compared to compound 1 in FIG. 5a (∘). It is possible that compound 4 has a different distribution profile when administered subcutaneously and may find a stable reservoir in a particular tissue or blood binding site.

### Subcutaneous Pharmacodynamics: COMT Inhibition

The pharmacodynamic properties of compounds 1 and 4 compared to oral administered micronized crystalline opicapone were assessed by subcutaneous injection to 4 male Wistar rats (3 mg/kg; DMSO:20% HPBCD (1:9)) then measurement of erythrocyte S-COMT activity over time.

During the studies, blood was collected at different time points, from tail vein, spun at 1500×g in a refrigerated centrifuge (4° C.) for 15 min, and the plasma and erythrocytes obtained were stored at −80° C. until further analysis. The plasma samples collected were analysed for compound 1, compound 4 and opicapone exposure, as previously described

Erythrocyte S-COMT activity was evaluated by the ability to methylate adrenaline to metanephrine. Aliquots of erythrocytes were haemolyzed by addition of 4 volumes of ice-cold ultrapure water and homogenization in a bead mill homogenizer, using glass beads, followed by 10 minutes incubation on wet ice. After centrifugation (20 000×g, 20 minutes, 4° C.), the supernatant containing S-COMT was quantified for total protein using Bradford assay. Supernatants were diluted with water to 4 mg total protein per ml. 100 μL of these diluted supernatants were then pre-incubated in a 96-well plate for 20 min with 80 μL of phosphate buffer (10 mM, pH 7.8); thereafter, the reaction mixture was incubated for 10 min with adrenaline (10 mM; 20 μL) in the presence of a saturating concentration of S-adenosyl-L-methionine, the methyl donor (500 μM). The incubation medium also contained pargyline (100 μM), MgCl2, (100 μM) and EGTA (1 mM). The pre-incubation and incubation were carried out at 37° C., under conditions of light protection with continuous shaking and without oxygenation. At the end of the incubation period the plate was transferred to ice and the reaction was stopped by the addition of 15 μL of glacial acetic acid. 60 μL of each sample were then transferred to a new 96-well containing 400 μL of 0.1% formic acid, followed by plate centrifugation (2000×g, 5 minutes 4° C.). The supernatants were used for the quantification of metanephrine by LC-MS/MS.

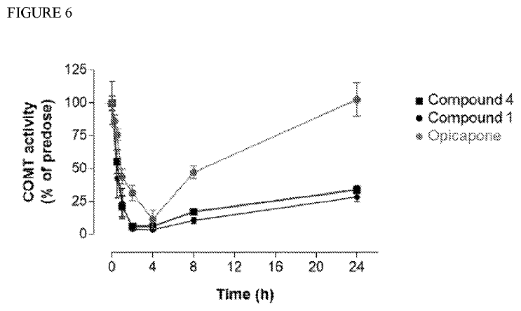

As shown in FIG. 6, conversion of compound 1 and compound 4 into opicapone produced a rapid inhibition of COMT activity in erythrocytes, with a maximal effect 2 h after dosing (96% and 94% inhibition for compound 1 and compound 4, respectively). Inhibition of COMT maintained above 80% at least until 8 h post-dosing, far longer than achieved with orally administered opicapone.

### Subcutaneous Pharmacodynamics: Impact on L-DOPA

As proof-of-concept, the effects of compound 4 on L-DOPA metabolism was evaluated in 4 male Wistar rats. Animals were subcutaneously injected with a single formulation (in DMSO:PEG 400:30% SBEBCD (1:2:2)) containing 12 mg/kg of L-DOPA plus 3 mg/kg of benserazide with or without 3 mg/kg of compound 4. Plasma samples were collected as described above and analyzed for levels of L-DOPA and its metabolite 3-O-methyldopa (3-OMD).

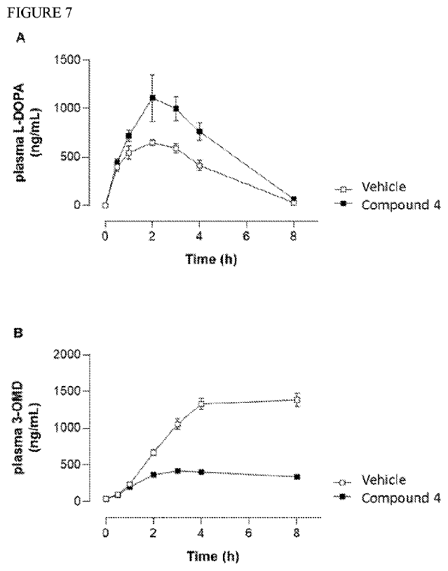

As shown in FIG. 7, concomitant administration of compound 4 with L-DOPA/benserazide increased circulating levels of L-DOPA (1.7-fold increase in the AUCₗₐₛₜ) with a corresponding decrease in the levels of its metabolite 3-OMD.

### SUMMARY

For both compound 1 and compound 4, the subcutaneous administration was the route where the conversion into opicapone was the highest, with a COMT inhibition above 80% at least until 8 hours post-dosing. When concomitantly administered with L-DOPA/benserazide, compound 4 showed an increase in L-DOPA levels and a decrease of 3-OMD level.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A compound of formula (I): 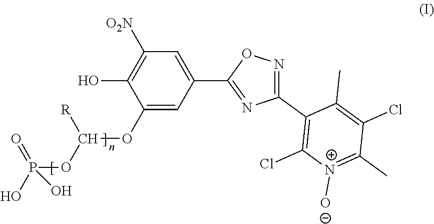
or a pharmaceutically acceptable salt thereof; wherein R is H or C₁-C₆ alkyl and n is 0 or 1.

2. The compound according to claim 1, wherein the pharmaceutically acceptable salt is selected from the group consisting of a sodium salt, an ammonium salt and a potassium salt.

3. The compound according to claim 2, wherein the pharmaceutically acceptable salt is selected from the group consisting of a sodium salt and an ammonium salt.

4. The compound according to claim 3, wherein the sodium salt is a disodium salt and wherein the ammonium salt is a triammonium salt.

5. A method of preparing a compound of formula (I), as defined in claim 1, or a pharmaceutically acceptable salt thereof, comprising:
(a) deprotecting a compound of formula (II) so as to provide a compound of formula (I): 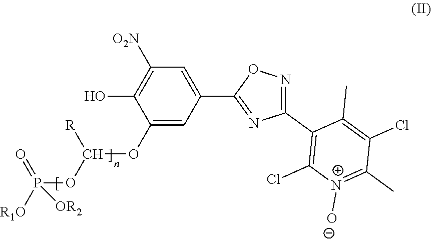
wherein R₁ and R₂ are each independently a monovalent phosphate protecting group or together form a divalent phosphate protecting group; R is H or C₁-C₆ alkyl and n is 0 or 1; and
(b) optionally converting the compound of formula (I) to a pharmaceutically acceptable salt thereof.

6. The method of claim 5, wherein the phosphate protecting groups R₁ and R₂ are each independently selected from the group consisting of C₁-C₆ alkyl and benzyl.

7. The method of claim 6, wherein the phosphate protecting groups R₁ and R₂ are both benzyl or both tert-butyl, or both ethyl.

8. The method of claim 5, wherein step (a) is carried out by reacting the compound of formula (II) with trimethylsilyl halide in an aprotic solvent, such as dichloromethane, or acetonitrile, followed by aqueous work up.

9. The method of claim 8, wherein the trimethylsilyl halide is trimethylsilyl bromide.

10. The method of claim 5, wherein the compound of formula (I) is converted to a pharmaceutically acceptable salt thereof.

11. The method of claim 10, wherein the pharmaceutically acceptable salt is selected from the group consisting of a sodium salt, an ammonium salt and a potassium salt.

12. The method of claim 11, wherein the pharmaceutically acceptable salt is selected from the group consisting of a sodium salt and an ammonium salt.

13. The method of claim 12, wherein the sodium salt is a disodium salt and wherein the ammonium salt is a triammonium salt.

14. A method of preparing a compound of formula (II), as defined in claim 5, comprising:
reacting a compound of formula (III): 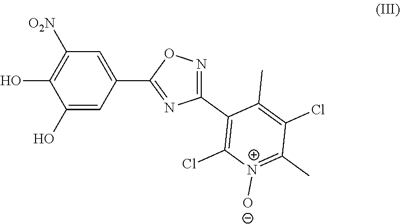 [SMILES: Cc1c(Cl)c(C)[n+]([O-])c(Cl)c1-c1noc(-c2cc(O)c(O)c([N+](=O)[O-])c2)n1]
with a compound of formula (IV): 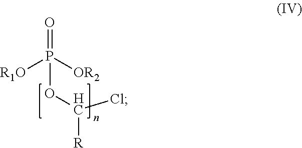
wherein R₁ and R₂ are each independently a monovalent phosphate protecting group or together form a divalent phosphate protecting group; R is H or C₁-C₆ alkyl and n is 0 or 1;
in the presence of a base and an aprotic solvent.

15. The method of claim 14, wherein the phosphate protecting groups R₁ and R₂ are each independently selected from the group consisting of C₁-C₆ alkyl and benzyl.

16. (canceled)

17. (canceled)

18. A compound of formula (II): 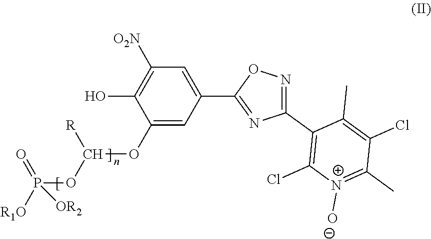
wherein R₁ and R₂ are each independently a monovalent phosphate protecting group or together form a divalent phosphate protecting group; R is H or C₁-C₆ alkyl and n is 0 or 1.

19. (canceled)

20. (canceled)

21. A pharmaceutical formulation comprising:
(i) a compound of formula (I), as defined in claim 1, or a pharmaceutically acceptable salt thereof; and
(ii) one or more pharmaceutically acceptable excipients.

22. A pharmaceutical formulation for intravenous administration to a human being comprising:
(i) a compound of formula (I), as defined in claim 1, or a pharmaceutically acceptable salt thereof; and
(ii) a pharmaceutically acceptable vehicle.

23. A pharmaceutical formulation for subcutaneous administration to a human being comprising:
(i) a compound of formula (I), as defined in claim 1, or a pharmaceutically acceptable salt thereof; and
(ii) a pharmaceutically acceptable vehicle.

24. (canceled)

25. (canceled)

26. (canceled)

27. (canceled)

28. A method of treating Parkinson's disease, comprising administering the compound of claim 1, or a pharmaceutically acceptable salt thereof, to a patent in need thereof.

29. (canceled)

30. (canceled)

31. (canceled)

32. (canceled)

33. (canceled)

34. (canceled)

35. (canceled)
